(19) United States
(12) Patent Application Publication
(10) Pub. No.: US 2026/0350270 A1
(43) Pub. Date: Nov. 5, 2026

(54) SIMULATION DEVICE, SIMULATION METHOD, AND PROGRAM

(71) Applicant: Resonac Corporation, Tokyo (JP)

(72) Inventors: Yuichiro ASOMA, Tokyo (JP); Teruo HIRAKAWA, Tokyo (JP); Yoshishige OKUNO, Tokyo (JP)

(21) Appl. No.: 19/487,850

(22) PCT Filed: Jul. 18, 2024
(86) PCT No.: PCT/JP2024/025834

§ 371 Date: Nov. 25, 2025

(30) Foreign Application Priority Data
Jul. 20, 2023 (JP) 2023-118565

(51) Int. Cl. G06F 30/27

(52) U.S. Cl. CPC G06F 30/27

(57) ABSTRACT

A simulation device includes: a selection unit configured to select a target coordinate in a structure space; an addition unit configured to add a potential to the target coordinate; a calculation unit configured to calculate a time evolution of the structure space in a first time interval by a first-principles calculation; and a prediction unit configured to predict a time evolution in a second time interval based on a prediction model that uses time as an explanatory variable and the target coordinate changed by applying an artificial force derived from the potential as a target variable.

### TECHNICAL FIELD

The present disclosure relates to a simulation device, simulation method, and program.

### BACKGROUND OF THE INVENTION

A technique for obtaining a free energy surface using molecular simulation is known. In this type of technique, a reaction path search method (metadynamics) is sometimes used to avoid falling into local minima. Metadynamics is a technique that enables global sampling by adding Gaussian function potentials to the sampled reaction coordinates, thereby preventing the already sampled reaction coordinates from being sampled again.

For example, Patent Document 1 discloses an invention in which metadynamics is used to search for stable binding structures, by employing as collective variables the dihedral angles formed between a target molecule and a drug candidate molecule, in order to efficiently explore multiple stable binding structures.

### RELATED-ART DOCUMENT

### Patent Document

Patent Document 1: WO 2019/130529

### SUMMARY OF THE INVENTION

### Problems to be Solved by the Invention

However, in the conventional technology, there is a problem that the computational cost for obtaining the free energy surface is large. For example, even when metadynamics is used, sampling is repeated in a narrow range in searching the vicinity of local minima, and the total amount of calculation until the free energy surface is obtained is large.

One aspect of the present disclosure is to obtain a free energy surface with low computational cost in view of the above technical problems.

### Means for Solving the Problems

The present disclosure includes the following configurations.

<1>

A simulation device including:
a selection unit configured to select a target coordinate in a structure space;
an addition unit configured to add a potential to the target coordinate;
a calculation unit configured to calculate a time evolution of the structure space in a first time interval by a first-principles calculation; and
a prediction unit configured to predict a time evolution in a second time interval based on a prediction model that uses time as an explanatory variable and the target coordinate changed by applying an artificial force derived from the potential as a target variable.
   <2>

The simulation device according to <1>, further including:
a learning unit configured to learn from a calculation result of the time evolution that has been calculated, thereby generating the prediction model.
   <3>

The simulation device according to <1> or <2>, further including:
a determination unit configured to determine whether to execute the calculation unit or the prediction unit in a next time interval, based on a difference between the calculation result of the time evolution that has been calculated and a prediction result of the time evolution that has been predicted in the same time interval.
   <4>

The simulation device according to <3>, wherein the determination unit is configured to be executed after the prediction unit predicts the time evolution in a predetermined number of time intervals.

<5>

The simulation device according to any one of <1> to <4>, wherein
the potential is a Gaussian function potential, and
the artificial force is calculated by a following equation, wherein b is a distance between a target coordinate in a current time interval and a target coordinate in a past time interval, and a and c are fitting functions

$${F}_{meta} = a \times exp ( - \frac{{b}^{2}}{{c}^{2}} ) . \qquad [ Math . 1 ]$$

<6> The simulation device according to <5>, wherein
the prediction model uses the target coordinate subjected to modulation by the artificial force as the target variable, and
the modulation is calculated by a following equation, wherein Fₘₑₜₐ is the artificial force, m is a mass of a particle forming a structure corresponding to the target coordinate, and Δt is a length of the time interval

$${R}_{m e t a} = \frac{{F}_{m e t a}}{m} \times \frac{1}{2} Δ {t}^{2} . \qquad [ Math . 2 ]$$

<7>

A simulation method, wherein a computer executes:
a procedure of selecting a target coordinate in a structure space;
a procedure of adding a potential to the target coordinate;
a procedure of calculating a time evolution of the structure space in a first time interval by a first-principles calculation; and
a procedure of predicting a time evolution in a second time interval based on a prediction model that uses time as an explanatory variable and the target coordinate changed by applying an artificial force derived from the potential as a target variable.
   <8>

A program for causing a computer to execute:
a procedure of selecting a target coordinate in a structure space;
a procedure of adding a potential to the target coordinate;
a procedure of calculating a time evolution of the structure space in a first time interval by a first-principles calculation; and
a procedure of predicting a time evolution in a second time interval based on a prediction model that uses time as an explanatory variable and the target coordinate changed by applying an artificial force derived from the potential as a target variable.

### Effects of the Invention

According to an aspect of the present disclosure, a free energy surface can be obtained with low computational cost.

### BRIEF DESCRIPTION OF THE DRAWINGS

FIG. 1 is a block diagram illustrating an example of an overall configuration of an information processing system.

FIG. 2 is a block diagram illustrating an example of a hardware configuration of a computer.

FIG. 3 is a block diagram illustrating an example of a functional configuration of an information processing system.

FIG. 4 is a flowchart illustrating an example of a simulation method.

### DETAILED DESCRIPTION OF THE INVENTION

Hereinafter, each embodiment of the present disclosure will be described with reference to the accompanying drawings. In the present specification and the drawings, components having substantially the same functional configuration are denoted by the same reference numerals and redundant descriptions thereof will be omitted.

### Embodiment

One embodiment of the present disclosure is an information processing system for executing molecular simulation. The information processing system according to the present embodiment includes a function of executing molecular simulation for obtaining a free energy surface.

There is a technique for calculating state quantities such as free energy by simulating particles such as atoms or molecules. In this type of technique, coordinates for calculating state quantities are sampled from the structure space, and the state quantities of the coordinates are repeatedly calculated by a first-principles calculation or the like. At this time, there is a possibility of falling into local minima because sufficient sampling cannot be performed due to limitations such as computational cost in terms of time or size.

Here, the structure space is a space including all structures having the positional relationship and bonding relationship of the particles constituting a substance. Each of the coordinates in the structure space corresponds to a single structure and indicate the conformation of the particles contained in the structure. An example of the structure space is the structure space of a reactive molecular system in which multiple molecules undergo chemical reactions.

One of the methods to prevent falling into local minima is a reaction path search method called metadynamics. In metadynamics, a potential based on a penalty function is added to the sampled coordinates, and the free energy surface is filled with the potential to avoid returning to the sampled coordinates. Metadynamics reduces the probability of falling into local minima and enables global sampling.

Gaussian functions are often used as the penalty function. Hereinafter, a potential based on a Gaussian function is also called a “Gaussian function potential”.

In metadynamics, when the free energy surface is filled with a potential, the system tends to shuttle between similar coordinates until escaping from the vicinity of that coordinate on the free energy surface. That is, in metadynamics, sampling near local minima is repeated in a narrow range. Therefore, to obtain the free energy surface using metadynamics, a large amount of computational cost is spent on sampling near local minima. Meanwhile, because there are few possible conformations of the particles near local minima, it is expected that the behavior of sampling can be predicted at high speed and with accuracy by learning the conformations.

An object of an embodiment of the present disclosure is to obtain a free energy surface with low computational cost. In the present embodiment, the time evolution of the structure space in a first time interval is calculated by a first-principles calculation, and the time evolution of the structure space in a second time interval is predicted based on a prediction model learned from the calculation result of the time evolution. In one aspect, according to the present embodiment, the free energy surface of a reactive molecular system can be obtained in a short time and with high accuracy.

### <Overall Configuration>

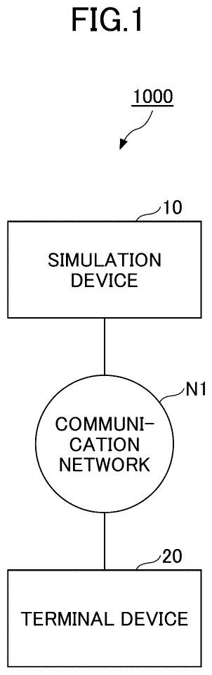

An overall configuration of the information processing system according to the present embodiment will be described with reference to FIG. 1. FIG. 1 is a block diagram illustrating an example of the overall configuration of the information processing system according to the present embodiment.

As illustrated in FIG. 1, an information processing system 1000 according to the present embodiment includes a simulation device 10 and a terminal device 20. The simulation device 10 and the terminal device 20 are connected to each other via a communication network N1 such as a local area network (LAN) or the Internet so as to enable data communication.

The simulation device 10 is an information processing device such as a personal computer, a workstation, or a server for executing molecular simulation. The simulation device 10 receives simulation conditions indicating conditions for executing molecular simulation from the terminal device 20. The simulation device 10 executes molecular simulation according to the simulation conditions and transmits the simulation result to the terminal device 20. In the present embodiment, the molecular simulation is, as an example, a simulation for obtaining a free energy surface in a reactive molecular system.

The terminal device 20 is an information processing terminal such as a personal computer, a smartphone, or a tablet terminal operated by a user of the information processing system 1000. The terminal device 20 transmits the simulation conditions input by the user to the simulation device 10. The terminal device 20 displays the simulation results received from the simulation device 10 to the user.

The overall configuration of the information processing system 1000 illustrated in FIG. 1 is an example, and there may be various system configuration examples in accordance with applications and purposes. For example, one or more simulation devices 10 and terminal devices 20 may be included in the information processing system 1000. For example, the simulation device 10 may be realized by a plurality of computers or may be realized as a cloud computing service. The classification of devices such as the simulation device 10 and the terminal device 20 illustrated in FIG. 1 is an example.

### <Hardware Configuration>

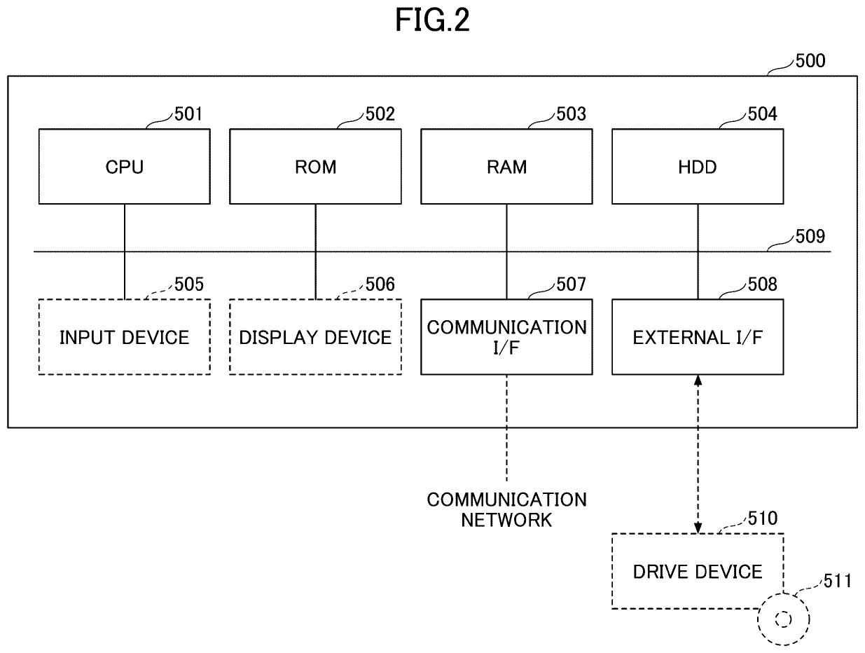

A hardware configuration of the information processing system 1000 according to the present embodiment will be described with reference to FIG. 2.

### <<Computer>>

The simulation device 10 and the terminal device 20 according to the present embodiment are realized by, for example, a computer. FIG. 2 is a block diagram illustrating an example of the hardware configuration of a computer 500 according to the present embodiment.

As illustrated in FIG. 2, the computer 500 includes a central processing unit (CPU) 501, a read only memory (ROM) 502, a random access memory (RAM) 503, a hard disk drive (HDD) 504, an input device 505, a display device 506, a communication interface (I/F) 507, and an external I/F 508. The CPU 501, the ROM 502, and the RAM 503 constitute what is known as a computer. The hardware components of the computer 500 are connected to each other via a bus line 509. The input device 505 and the display device 506 may be connected to the external I/F 508 for use.

The CPU 501 is an arithmetic unit that reads programs and data from a storage device such as the ROM 502 or the HDD 504 into the RAM 503 and executes processing, thereby realizing control and functions of the entire computer 500.

The ROM 502 is an example of a nonvolatile semiconductor memory (storage device) that can hold programs and data even when the power is turned off. The ROM 502 functions as a main storage device for storing various programs and data necessary for the CPU 501 to execute various programs installed in the HDD 504. Specifically, the ROM 502 stores a boot program such as basic input/output system (BIOS) or extensible firmware interface (EFI) to be executed when the computer 500 is started, and data on operating system (OS) settings, network settings, and the like.

The RAM 503 is an example of a volatile semiconductor memory (storage device) from which programs and data are erased when the power is turned off. The RAM 503 is, for example, dynamic random access memory (DRAM) or static random access memory (SRAM). The RAM 503 provides a work area to be expanded when various programs installed in the HDD 504 are executed by the CPU 501.

The HDD 504 is an example of a nonvolatile storage device for storing programs and data. The programs and data stored in the HDD 504 include an OS, which is basic software for controlling the entire computer 500, and applications for providing various functions on the OS. Instead of the HDD 504, the computer 500 may use a storage device (for example, solid state drive (SSD) and the like) using a flash memory as a storage medium.

The input device 505 includes a touch panel used by a user to input various signals, operation keys and buttons, a keyboard and mouse, a microphone for inputting sound data such as voice, and the like.

The display device 506 includes a liquid crystal display or an organic electro-luminescence (EL) display for displaying a screen, a speaker for outputting sound data such as voice, and the like.

The communication I/F 507 is an interface for connecting to a communication network and allowing the computer 500 to perform data communication.

The external I/F 508 is an interface with an external device. The external device includes a drive device 510 and the like.

The drive device 510 is a device for setting the recording medium 511. Here, the recording medium 511 includes a medium for optically, electrically, or magnetically recording information such as a CD-ROM, a flexible disk, a magneto-optical disk, or the like. Further, the recording medium 511 may include a semiconductor memory for electrically recording information such as a ROM, a flash memory, or the like. Thus, the computer 500 can read and/or write the recording medium 511 via the external I/F 508.

The various programs installed in the HDD 504 are installed by, for example, setting the distributed recording medium 511 in the drive device 510 connected to the external I/F 508 and reading the various programs recorded in the recording medium 511 by the drive device 510. Alternatively, the various programs installed in the HDD 504 may be installed by being downloaded from another network different from the communication network via the communication I/F 507.

### <Functional Configuration>

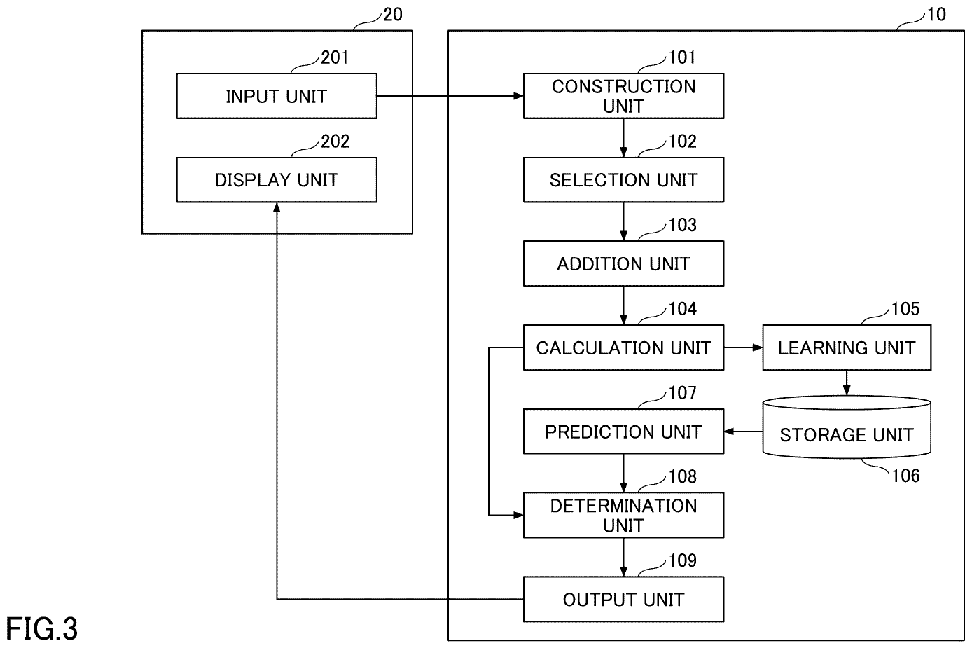

A functional configuration of the information processing system according to the present embodiment will be described with reference to FIG. 3. FIG. 3 is a block diagram illustrating an example of the functional configuration of the information processing system according to the present embodiment.

### <<Simulation Device>>

As illustrated in FIG. 3, the simulation device 10 according to the present embodiment includes a construction unit 101, a selection unit 102, an addition unit 103, a calculation unit 104, a learning unit 105, a storage unit 106, a prediction unit 107, a determination unit 108, and an output unit 109.

The construction unit 101, the selection unit 102, the addition unit 103, the calculation unit 104, the learning unit 105, the prediction unit 107, the determination unit 108, and the output unit 109 are realized by a process executed by the CPU 501 by a program loaded into the RAM 503 from the HDD 504 illustrated in FIG. 2. The storage unit 106 is realized by the HDD 504 illustrated in FIG. 2.

The construction unit 101 receives a simulation condition from the terminal device 20. The simulation condition includes information indicating a plurality of substances causing a desired chemical reaction. The construction unit 101 constructs the structure space of the reactive molecular system according to the simulation conditions.

The selection unit 102 selects a reaction coordinate as a calculation object of the state quantity from the structure space constructed by the construction unit 101. In the present embodiment, as an example, the state quantity is a free energy. Hereinafter, the reaction coordinate as a calculation object of the free energy is also referred to as an “target coordinate”.

The addition unit 103 adds a potential to the target coordinate selected by the selection unit 102. In the present embodiment, as an example, a Gaussian function potential is added. The Gaussian function potential to be added to the target coordinate is generated based on a structure corresponding to the target coordinate.

The calculation unit 104 calculates the time evolution of the structure space by a first-principles calculation. In the present embodiment, as an example, the first-principles calculation is a calculation method based on the density functional theory (DFT). The calculation unit 104 calculates the time evolution in a predetermined time interval. The length of the time interval corresponds to the time step size in the molecular simulation. The time step size may be freely set.

In the present embodiment, the calculation unit 104 applies an artificial force to the target coordinate in the previous time interval to change it to the target coordinate in the next time interval. The artificial force is derived from the Gaussian function potential added to the structure space by the addition unit 103.

Specifically, the artificial force is Fₘₑₜₐ defined by an equation (1). The artificial force Fₘₑₜₐ is applied to each particle included in the structure corresponding to the target coordinate. For simplicity, the index indicating the particle is omitted from the equation (1), but the artificial force Fₘₑₜₐ is calculated for each particle.

$$\begin{matrix}[ Math . 3 ] & \\ {F}_{m e t a} = a \times exp ( - \frac{{b}^{2}}{{c}^{2}} ) & ( 1 )\end{matrix}$$

In the equation, b is the distance between the target coordinate in the current time interval and the target coordinate in the past time interval, and a and c are fitting functions.

When the artificial force Fₘₑₜₐ is applied to the target coordinate, the target coordinate receives a modulation Rₘₑₜₐ defined by an equation (2). Because the artificial force Fₘₑₜₐ is applied to each particle, the modulation Rₘₑₜₐ is also calculated for each particle. In the equation (2), as in the equation (1), the index indicating the particle is omitted.

$$\begin{matrix}[ Math . 4 ] & \\ {F}_{m e t a} = \frac{{F}_{meta}}{m} \times \frac{1}{2} Δ {t}^{2} & ( 2 )\end{matrix}$$

In the equation, Fₘₑₜₐ is the artificial force, m is the mass of the particle, and Δt is the length of the time interval (time step size).

The learning unit 105 generates a prediction model by learning from the calculation result of the time evolution calculated by the calculation unit 104. When there is a learned prediction model, the learning unit 105 updates the prediction model by learning from the calculation result of the time evolution newly calculated by the calculation unit 104.

The prediction model is a machine learning model that uses time as an explanatory variable and a coordinate as a target variable. The coordinate output by the prediction model is the target coordinate sampled at the input time. The target coordinate calculated by the calculation unit 104 is the target coordinate subjected to the modulation Rₘₑₜₐ defined by the equation (2) by adding the artificial force Fₘₑₜₐ calculated by the equation (1). Therefore, the coordinate output by the prediction model is the predicted value of the target coordinate subjected to the modulation Rₘₑₜₐ by adding the artificial force Fₘₑₜₐ to the target coordinate in the previous time interval.

The structure of the prediction model can be freely selected. For example, molecular dynamics-generative adversarial network (MD-GAN) disclosed in Reference 1 can be used. MD-GAN is a multilayer neural network capable of predicting the time evolution in molecular dynamics calculation in the framework of generative adversarial networks.
[Reference 1] Katsuhiro Endo, Katsufumi Tomobe, Kenji Yasuoka, “Multi-Step Time Series Generator for Molecular Dynamics,” Thirty-Second AAAI Conference on Artificial Intelligence, Vol. 32, No. 1 (2018)

The storage unit 106 stores the learned prediction model. The prediction model stored in the storage unit 106 is generated or updated by the learning unit 105.

The prediction unit 107 predicts the time evolution of the structure space based on the prediction model read from the storage unit 106. The prediction unit 107 predicts the time evolution in a predetermined time interval. The time interval in which the prediction unit 107 predicts the time evolution is different from the time interval in which the time evolution is calculated by the calculation unit 104. The length of the time interval may be the same as the length of the time interval in which the calculation unit 104 calculates the time evolution. Therefore, the prediction unit 107 inputs the time (hereinafter also referred to as “target time”) when the predetermined time interval has elapsed from the current time to the prediction model. The prediction unit 107 obtains the prediction result including the target coordinate output from the prediction model.

The determination unit 108 determines whether to execute the calculation unit 104 or the prediction unit 107 based on the calculation result of the time evolution calculated by the calculation unit 104 and the prediction result of the time evolution predicted by the prediction unit 107. The determination unit 108 determines whether to execute the calculation unit 104 or the prediction unit 107 in the next time interval by comparing the error between the calculation result and the prediction result in the same time interval with a predetermined threshold value.

The output unit 109 determines whether or not the end condition of the simulation is satisfied. In the present embodiment, the end condition is that a desired chemical reaction has occurred. That is, the output unit 109 determines whether or not the desired chemical reaction has occurred in the current structure space.

When the end condition of the simulation is satisfied, the output unit 109 outputs a simulation result. The simulation result includes state information indicating the state of the current structure space. The state information is, for example, information indicating a free energy surface.

### <<Terminal Device>>

As illustrated in FIG. 3, the terminal device 20 according to the present embodiment includes an input unit 201 and a display unit 202.

The input unit 201 and the display unit 202 are realized by a process in which a program expanded from the HDD 504 onto the RAM 503 illustrated in FIG. 2 causes the CPU 501 to execute.

The input unit 201 receives an input of a simulation condition in response to a user operation. The input unit 201 transmits the simulation condition to the simulation device 10.

The display unit 202 receives a simulation result from the simulation device 10. The display unit 202 outputs the simulation result to the display device 506 or the like.

### <Processing Procedure>

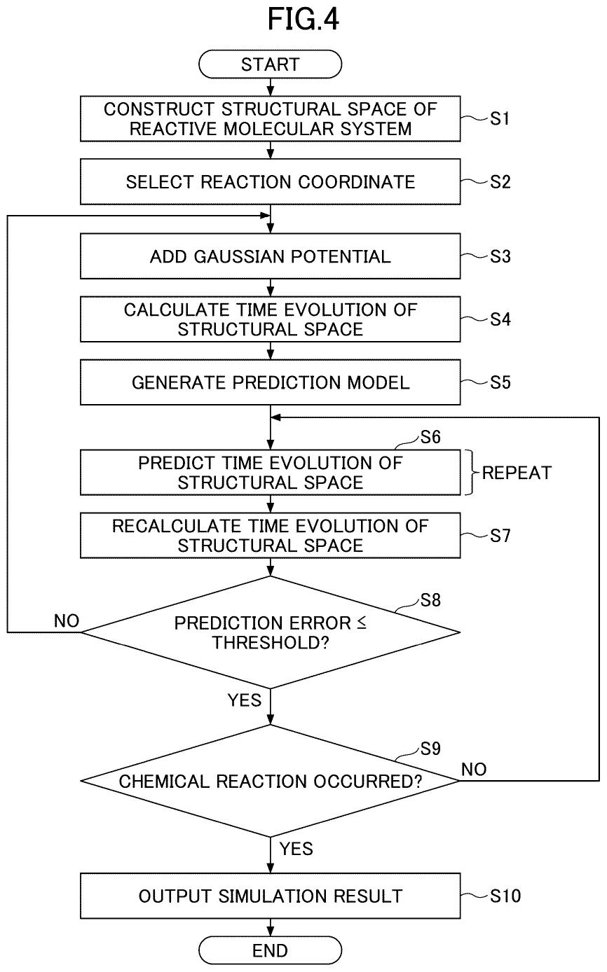

A simulation method executed by the information processing system 1000 according to the present embodiment will be described with reference to FIG. 4. FIG. 4 is a flowchart illustrating an example of the simulation method according to the present embodiment.

In step S1, the input unit 201 of the terminal device 20 receives an input of a simulation condition in response to a user operation. The simulation condition includes information indicating a plurality of substances causing a chemical reaction desired by the user. Next, the input unit 201 transmits the simulation condition to the simulation device 10.

In the simulation device 10, the construction unit 101 receives the simulation condition from the terminal device 20. Next, the construction unit 101 constructs the structure space of the reactive molecular system according to the simulation condition. Then, the construction unit 101 sends the constructed structure space to the selection unit 102.

In step S2, the selection unit 102 of the simulation device 10 receives the structure space from the construction unit 101. Next, the selection unit 102 selects the target coordinate from the structure space by using metadynamics. Then, the selection unit 102 sends the structure space in which the target coordinate is selected to the addition unit 103.

In step S3, the addition unit 103 of the simulation device 10 receives the structure space from the selection unit 102. Next, the addition unit 103 generates a Gaussian function potential based on the structure corresponding to the target coordinate. Subsequently, the addition unit 103 adds the Gaussian function potential to the target coordinate. Then, the addition unit 103 sends, to the calculation unit 104, the structure space obtained by adding the Gaussian function potential to the target coordinate.

In step S4, the calculation unit 104 of the simulation device 10 receives the structure space from the addition unit 103. Next, the calculation unit 104 calculates the time evolution of the structure space in the next time interval by the first-principles calculation. Then, the calculation unit 104 sends the calculation result of the time evolution to the learning unit 105.

Specifically, the calculation unit 104 first derives the artificial force Fₘₑₜₐ from the Gaussian function potential added in step S3 according to the equation (1). Next, the calculation unit 104 calculates the modulation Rₘₑₜₐ to which the target coordinate is subjected by applying the artificial force Fₘₑₜₐ according to the equation (2). Subsequently, the calculation unit 104 changes the structure corresponding to the target coordinate based on the modulation Rₘₑₜₐ and selects the target coordinate corresponding to the structure after the change.

The calculation unit 104 repeatedly executes step S4 until a sufficient amount of calculation results for generating a prediction model is accumulated. The sufficient amount for generating the prediction model is predetermined in accordance with the type of the prediction model. When the sufficient amount of calculation results is accumulated, the calculation unit 104 sends the structure space to the prediction unit 107.

In step S5, the learning unit 105 of the simulation device 10 receives the calculation results of the time evolution from the calculation unit 104. Next, the learning unit 105 learns from the calculation results of the time evolution to generate a prediction model that uses time as an explanatory variable and a coordinate as a target variable. Then, the learning unit 105 stores the learned prediction model in the storage unit 106.

In step S6, the prediction unit 107 of the simulation device 10 receives the structure space from the calculation unit 104. Next, the prediction unit 107 reads the learned prediction model from the storage unit 106.

Subsequently, the prediction unit 107 calculates the target time by adding the length of the next time interval to the current time. Next, the prediction unit 107 inputs the target time to the learned prediction model. The prediction model predicts the target coordinate at the inputted target time. At this time, a Gaussian function potential based on the structure corresponding to the target coordinate is added to the target coordinate. The prediction model outputs a predicted value of the target coordinate. The prediction unit 107 obtains the prediction result outputted by the prediction model. The prediction result includes the target coordinate when the next time interval passes.

The prediction unit 107 repeats step S6 until the time evolution in a predetermined number of time intervals is predicted. The number of repetitions may be freely set, but may be 100 times, for example. When the prediction unit 107 predicts the time evolution in a predetermined number of time intervals, the prediction result of the time evolution is sent to the determination unit 108.

In step S7, the calculation unit 104 of the simulation device 10 calculates the time evolution of the structure space in the current time interval by the first-principles calculation. In other words, the calculation unit 104 calculates the time evolution in the current time interval based on the prediction result of the time evolution in the immediately preceding time interval. The calculation unit 104 sends the calculation result of the time evolution to the determination unit 108.

In step S8, the determination unit 108 of the simulation device 10 receives the prediction result of the time evolution in the current time interval from the prediction unit 107. The determination unit 108 also receives the calculation result of the time evolution in the current time interval from the calculation unit 104.

Next, the determination unit 108 compares the prediction result of the time evolution in the current time interval with the calculation result. Subsequently, the determination unit 108 determines whether the prediction error is equal to or less than a predetermined threshold value. The prediction error is a distance between the target coordinate indicated by the prediction result and the target coordinate indicated by the calculation result. That is, the determination unit 108 determines whether the target coordinate predicted by the prediction model in the current time interval significantly deviates from the target coordinate calculated by the first-principles calculation.

When the prediction error is equal to or less than the threshold value (YES), the determination unit 108 sends the structure space to the output unit 109 and causes the process to proceed to step S9. When the prediction error is greater than the threshold value (NO), the determination unit 108 sends the structure space to the addition unit 103 and causes the process to return to step S3.

In step S9, the output unit 109 of the simulation device 10 receives the structure space from the determination unit 108. Next, the output unit 109 determines whether or not a desired chemical reaction has occurred based on the structure space. When the chemical reaction has occurred (YES), the output unit 109 causes the process to proceed to step S10. When the chemical reaction has not occurred (NO), the output unit 109 causes the process to return to step S6.

In step S10, the output unit 109 of the simulation device 10 outputs the simulation result. The simulation result includes information indicating the free energy surface. The output unit 109 transmits the simulation result to the terminal device 20.

In the terminal device 20, the display unit 202 receives the simulation result from the simulation device 10. Next, the display unit 202 outputs the simulation result to the display device 506. The user can refer to the simulation result outputted to the display device 506 of the terminal device 20 and use the calculation result of the free energy surface.

For example, the user can use the calculation result of the free energy surface to search for a raw material in the reactive molecular system. Further, for example, the user may acquire a measured value of the actual reactive molecular system by experiment or manufacturing, update the prediction model based on the simulation result and the measured value, and repeatedly execute the search and prediction of the reaction path.

After returning to step S3 from step S8, the simulation device 10 executes the processing from step S3 to step S8 again. In step S5 from the second time onwards, the learned prediction model is updated based on the new calculation result calculated in step S4 immediately before. In contrast, after returning to step S6 from step S9, the simulation device 10 executes the processing from step S6 to step S8 again. That is, the determination unit 108 determines whether to execute the calculation unit 104 or the prediction unit 107 in the next time interval based on the error between the calculation result and the prediction result in the same time interval.

In this manner, the simulation device 10 predicts the time evolution of the structure space at high speed by the prediction model as long as the prediction error by the prediction model is equal to or less than the threshold value, and calculates the time evolution of the structure space with high accuracy by the first-principles calculation when the prediction error exceeds the threshold value. Thus, the simulation device 10 can calculate the time evolution of the structure space at high speed and with high accuracy, and can acquire the free energy surface at high speed and with high accuracy.

### Effect of Embodiment

The simulation device 10 according to the present embodiment adds the potential to the target coordinate in the structure space, calculates the time evolution of the structure space in the first time interval by the first-principles calculation, and predicts the time evolution of the structure space in the second time interval based on the prediction model. The prediction model uses time as the explanatory variable and the target coordinate changed by applying the artificial force derived from the potential as the target variable. The simulation device 10 can reduce the number of the first-principles calculations by predicting the time evolution of the structure space in a part of the time interval. Therefore, according to the present embodiment, the free energy surface can be obtained with low computational cost.

The simulation device 10 generates the prediction model by learning from the calculation result of the time evolution. The simulation device 10 updates the prediction model every time the time evolution is calculated by the first-principles calculation. Therefore, according to the present embodiment, the time evolution of the structure space can be predicted with high accuracy.

The simulation device 10 determines whether to execute the first-principles calculation or the prediction model in the next time interval based on the difference between the calculation result of the time evolution and the prediction result of the time evolution in the same time interval. When the target coordinate is largely moved by the time evolution, the accuracy of the prediction result may be lowered. Therefore, according to the present embodiment, the free energy surface can be obtained with high accuracy.

The simulation device 10 calculates the time evolution by the first-principles calculation after predicting the time evolution in a predetermined number of time intervals. The simulation device 10 can reduce the computational cost as the number of times of executing the prediction model increases. Therefore, according to the present embodiment, the free energy surface can be obtained with significantly reduced computational cost.

### [Note]

Each of the functions of the embodiments described above can be implemented by one or more processing circuits. Here, the term “processing circuit” as used herein includes a processor programmed to execute each of the functions by software, such as a central processing unit (CPU) or a graphics processing unit (GPU) implemented by an electronic circuit, and devices such as an application specific integrated circuit (ASIC), a digital signal processor (DSP), a field programmable gate array (FPGA), and conventional circuit modules designed to execute each of the functions described above.

Although the embodiments of the present disclosure have been described in detail above, the embodiments disclosed herein are exemplary and not restrictive in all respects. The embodiments may be modified and improved in various ways without departing from the scope of the appended claims. The matters described in the above embodiments may be adopted in other configurations to the extent that they do not conflict, and may also be combined with each other within a non-conflicting scope.

The present international application claims priority to Japanese Patent Application No. 2023-118565 filed Jul. 20, 2023, the entire contents of which are incorporated herein by reference.

### DESCRIPTION OF REFERENCE NUMERALS

10 Simulation device
20 Terminal device
101 Construction unit
102 Selection unit
103 Addition unit
104 Calculation unit
105 Learning unit
106 Storage unit
107 Prediction unit
108 Determination unit
109 Output unit
201 Input unit
202 Display unit
1000 Information processing system

1. A simulation device comprising:
a memory; and
a processor coupled to the memory and the processor is configured to:
select a target coordinate in a structure space;
add a potential to the target coordinate;
calculate a time evolution of the structure space in a first time interval by a first-principles calculation; and
predict a time evolution in a second time interval based on a prediction model that uses time as an explanatory variable and the target coordinate changed by applying an artificial force derived from the potential as a target variable.

2. The simulation device according to claim 1, wherein the processor is further configured to:
learn from a calculation result of the time evolution that has been calculated, thereby generating the prediction model.

3. The simulation device according to claim 1, wherein the processor is further configured to:
determine whether to execute the calculation or the prediction in a next time interval, based on a difference between the calculation result of the time evolution that has been calculated and a prediction result of the time evolution that has been predicted in the same time interval.

4. The simulation device according to claim 3, wherein the determination is executed after the prediction of the time evolution in a predetermined number of time intervals.

5. The simulation device according to claim 1, wherein
the potential is a Gaussian function potential, and
the artificial force is calculated by a following equation, wherein b is a distance between a target coordinate in a current time interval and a target coordinate in a past time interval, and a and c are fitting functions
$${F}_{m e t a} = a \times exp ( - \frac{{b}^{2}}{{c}^{2}} ) .$$

6. The simulation device according to claim 5, wherein
the prediction model uses the target coordinate subjected to modulation by the artificial force as the target variable, and
the modulation is calculated by a following equation, wherein Fₘₑₜₐ is the artificial force, m is a mass of a particle forming a structure corresponding to the target coordinate, and Δt is a length of the time interval
$${R}_{m e t a} = \frac{{F}_{m e t a}}{m} \times \frac{1}{2} Δ {t}^{2} .$$

7. A simulation method, wherein a computer executes:
selecting a target coordinate in a structure space;
adding a potential to the target coordinate;
calculating a time evolution of the structure space in a first time interval by a first-principles calculation; and
predicting a time evolution in a second time interval based on a prediction model that uses time as an explanatory variable and the target coordinate changed by applying an artificial force derived from the potential as a target variable.

8. A non-transitory computer-readable recording medium storing a program configured for causing a computer to execute:
selecting a target coordinate in a structure space;
adding a potential to the target coordinate;
calculating a time evolution of the structure space in a first time interval by a first-principles calculation; and
predicting a time evolution in a second time interval based on a prediction model that uses time as an explanatory variable and the target coordinate changed by applying an artificial force derived from the potential as a target variable.
